(19) United States
(12) Patent Application Publication
(10) Pub. No.: US 2025/0369944 A1
(43) Pub. Date: Dec. 4, 2025

(54) SYSTEMS AND METHODS FOR AIR QUALITY IMPACT MONITORING IN TRAFFIC MONITORING SYSTEMS

(71) Applicant: Rekor Systems, Inc.

(72) Inventor: Christopher Allen KADOCH, Columbia, MD (US)

(21) Appl. No.: 19/220,166

(22) Filed: May 28, 2025

Related U.S. Application Data
(60) Provisional application No. 63/652,981, filed on May 29, 2024.

(51) Int. Cl. G01N 33/00; G08G 1/01

(52) U.S. Cl. CPC G01N 33/0075; G08G 1/0125

(57) ABSTRACT

A traffic monitoring system includes one or more traffic sensors configured to: capture images of vehicles on a road segment, generate traffic related data from the captured images, and transmit the traffic related data to a server. The server is configured to: receive the traffic related data from the one or more traffic sensors, analyze the traffic related data so as to generate an air quality index impact metric quantifying a change to an air quality index as indicated by the traffic related data.

### CROSS-REFERENCE TO RELATED APPLICATIONS

This application claims the benefit of U.S. Provisional Application No. 63/652,981, filed May 29, 2024, the disclosure of which are expressly incorporated by reference herein.

### BACKGROUND

The present invention relates to traffic monitoring systems and methods, and more particularly to monitoring air quality impact via such systems and methods.

Motor vehicles travelling on roadways are a significant source of air quality affecting emissions. It is therefore desirable to monitor the impact of such motor vehicles on air quality, particularly on localized air quality. Traditionally, air quality affecting emissions are measured during a dedicated “smog-testing” session via a probe inserted into a stationary running motor vehicle. While highly accurate, such testing is inconvenient and inefficient. More recently, spectrophotometric sensors have been used to monitor air quality affecting emissions of vehicles in motion. However, such sensors are expensive, inefficient, and difficult to deploy on large scales.

At the same time, traffic monitoring systems generally monitor vehicle traffic on large scales. Such systems generally include traffic cameras that capture video clips of passing traffic—e.g., roadway traffic—for playback review by law enforcement or other interested users.

It is therefore desirable to provide a traffic monitoring system that monitors air quality affecting emissions via captured video clips.

### BRIEF SUMMARY OF THE INVENTION

Systems and methods are disclosed for a traffic monitoring system that monitors air quality affecting emissions via captured video clips.

Other objects, advantages and novel features of the present invention will become apparent from the following detailed description of one or more preferred embodiments when considered in conjunction with the accompanying drawings. It should be recognized that the one or more examples in the disclosure are non-limiting examples and that the present invention is intended to encompass variations and equivalents of these examples.

### BRIEF DESCRIPTION OF THE DRAWINGS

The features, objects, and advantages of the present invention will become more apparent from the detailed description, set forth below, when taken in conjunction with the drawings, in which like reference characters identify elements correspondingly throughout.

FIG. 1 illustrates an exemplary system in accordance with at least one embodiment;

FIG. 2 illustrates an exemplary method in accordance with at least one embodiment; and

FIG. 3 illustrates an exemplary interactive heat map in accordance with at least one embodiment.

### DETAILED DESCRIPTION OF THE PREFERRED EMBODIMENTS

The above described drawing figures illustrate the present invention in at least one embodiment, which is further defined in detail in the following description. Those having ordinary skill in the art may be able to make alterations and modifications to what is described herein without departing from its spirit and scope. While the present invention is susceptible of embodiment in many different forms, there is shown in the drawings and will herein be described in detail at least one preferred embodiment of the invention with the understanding that the present disclosure is to be considered as an exemplification of the principles of the present invention, and is not intended to limit the broad aspects of the present invention to any embodiment illustrated.

In accordance with the practices of persons skilled in the art, the invention is described below with reference to operations that are performed by a computer system or a like electronic system. Such operations are sometimes referred to as being computer-executed. It will be appreciated that operations that are symbolically represented include the manipulation by a processor, such as a central processing unit, of electrical signals representing data bits and the maintenance of data bits at memory locations, such as in system memory, as well as other processing of signals. The memory locations where data bits are maintained are physical locations that have particular electrical, magnetic, optical, or organic properties corresponding to the data bits.

When implemented in software, code segments perform certain tasks described herein. The code segments can be stored in a processor readable medium. Examples of the processor readable mediums include an electronic circuit, a semiconductor memory device, a read-only memory (ROM), a flash memory or other non-volatile memory, a floppy diskette, a CD-ROM, an optical disk, a hard disk, etc.

In the following detailed description and corresponding figures, numerous specific details are set forth in order to provide a thorough understanding of the present invention. However, it should be appreciated that the invention may be practiced without such specific details. Additionally, well-known methods, procedures, components, and circuits have not been described in detail.

The present invention generally relates to traffic monitoring systems and methods, and more particularly to such systems and methods for monitoring air quality affecting emissions via captured video clips.

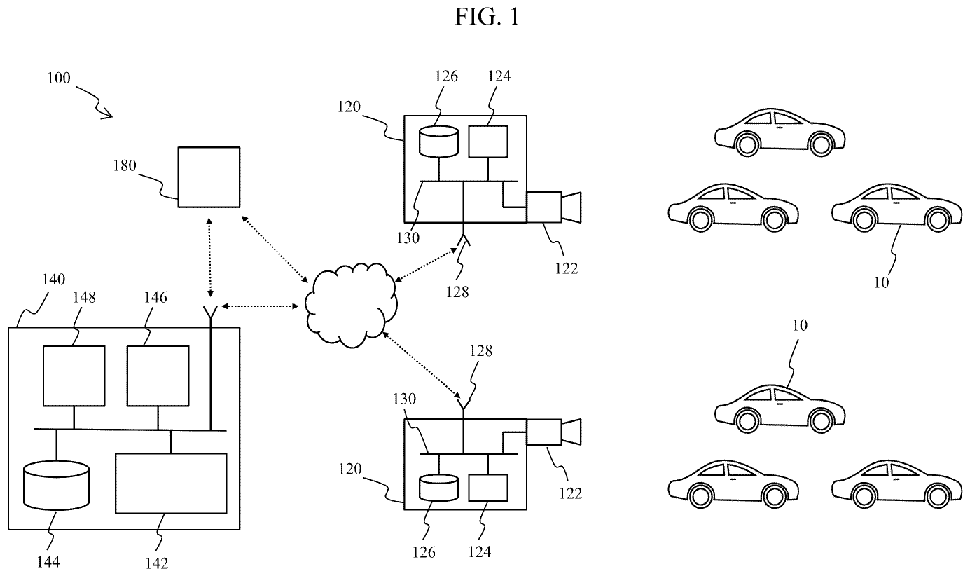

FIG. 1 is a schematic representation of a traffic monitoring system 100 in accordance with one or more aspects of the invention. As shown in FIG. 1, the traffic monitoring system 100 comprises one or more traffic sensors 120 communicatively coupled to a system server 140, via a network 160. The system server may also be communicatively coupled to one or more user devices 180 via the network 160. The traffic monitoring system 100 generally enables the collection of traffic related data for transmission, via the network 106, to the system server 140. The traffic monitoring system 100 also generally enables user access to the traffic related data stored on the system server 140, via the coupled user devices 180.

The traffic sensors 120 may each comprise an imaging device 122, a controller 124, a memory 126, and a transceiver 128, each communicatively coupled to a common data bus 130 that enables data communication between the respective components.

The imaging device 122 may capture images of traffic, in particular, video images of vehicles 10 making up the traffic, and generates video data therefrom. The imaging device 122 may be a video camera of any camera type, which captures video images suitable for computerized image recognition of objects within the captured images. For example, the camera may utilize charge-coupled-device (CCD), complementary metal-oxide-semiconductor (CMOS) and/or other imaging technology, to capture standard, night-vision, infrared, and/or other types of images, having predetermined resolution, contrast, color depth, and/or other image characteristics. The video data may be timestamped so as to indicate the date and time of recording. The video data may further include other identifying information, including geolocation data and/or traffic sensor ID data. It will be understood that, while the invention is described herein with respect to video data, still image data may be similarly used, or may be otherwise generated from the video data, without departing from the scope of the invention.

The controller 124 may be generally configured to control the imaging device 122, the memory 126, and the transceiver 128, in accordance with the functions described herein. In at least one embodiment, the controller 124 may execute image processing software for applying image processing to the video data captured by the imaging device 122 so as to generate processed video data. Some exemplary types of image processing that may be applied to the video data include image enhancement, encoding, compression, and recognition processing.

In some embodiments, the controller 124 may also generate one or more recognition records from the processed video data. The recognition records are datasets comprising one or more values reflecting image recognized vehicle and/or traffic characteristics. These characteristic values may be associated with corresponding confidence scores indicating the confidence with which the particular characteristic value was determined.

Accordingly, in some embodiments, the controller 124 may apply computerized image recognition techniques to identify objects within the video images. For example, the controller 124 may identify individual vehicles captured by the video images, as well as their associated characteristics. These vehicle characteristics may include, for example, vehicle type, class, make, model, color, year, drive type (e.g., electric, hybrid, etc.), license plate number, registration, trajectory, speed, location, etc., or any combination thereof. The controller 124 may also apply image analysis techniques to the image-recognized video images so as to identify captured traffic characteristics. These traffic characteristics may include, for example, temporal histories, vehicle counts, congestion levels, the presence of accidents, disabled vehicles, foreign objects, or other traffic incidents, or any combination thereof.

The recognition record may also include some or all of the processed video data. The included processed video data may be low-resolution or low-bit video data and/or limited frame video data. That is, the included processed video data may be of lower resolution/bit and/or more limited in frames than the source video data from which it is generated. In some embodiments, the recognition record comprises the processed video data having metadata that includes the dataset with one or more of the characteristic values discussed herein.

The recognition record may still also include the identifier (e.g., timestamp, geolocation data, sensor ID, etc.) associated with the corresponding source video data from which it was generated. Accordingly, the identifier may be used to identify the source video data corresponding to the recognition record.

The controller 124 may be embodied, collectively or individually, as one or more processors programmed to carry out the functions described herein in accordance with software stored in the memory 126. Each processor may be a standard processor, such as a central processing unit (CPU), or a dedicated processor, such as an application-specific integrated circuit (ASIC) or field programable gate array (FPGA), or portion thereof.

The memory 126 stores software and data that can be accessed by the processor(s), and includes both transient and persistent storage. The transient storage is configured to temporarily store data being processed or otherwise acted on by other components, and may include a data cache, RAM or other transient storage types. The persistent storage is configured to store software and data until deleted. The memory 126 is accordingly configured to store the software, data and information described herein.

The transceiver 128 communicatively couples the traffic sensor 120 to the network 160 so as to enable data transmission therewith. In particular, the transceiver 128 may be configured to transmit the processed video data and/or the recognition records to the system server 140 via the network 160.

The network 160 may be any type of network, wired or wireless, configured to facilitate the communication and transmission of data, instructions, etc., and may include a local area network (LAN) (e.g., Ethernet or other IEEE 802.03 LAN technologies), Wi-Fi (e.g., IEEE 802.11 standards, wide area network (WAN), virtual private network (VPN), global area network (GAN)), a cellular network, or any other type of network or combination thereof.

The system server 140 may include one or more server computers connected to the network. Each server computer may include computer components, including one or more processors, memories, displays and interfaces, and may also include software instructions and data for executing the functions of the server described herein. The servers may also include one or more storage devices 144 configured to store large quantities of data and/or information, and may further include one or more databases. For example, the storage device may be a collection of storage components, or a mixed collection of storage components, such as ROM, RAM, hard-drives, solid-state drives, removable drives, network storage, virtual memory, cache, registers, etc., configured so that the server computers may access it. The storage devices may also support one or more databases for the storage of data therein.

The system server 140 is generally configured to provide centralized support for the traffic sensors 120. The system server 140 is configured to receive traffic sensor generated data (e.g., video and other data) from each of the traffic sensors 120, and to store the data for users to access via the user devices 180. The system server 140 may therefore include one or more databases configured to store the data received from the traffic sensors 120.

In at least one embodiment, the system server 140 comprises an analysis engine 142 (not shown), which may be generally configured to analyze the traffic sensor generated data to generate vehicle and/or traffic metrics for various periods of time. The metrics may be generated via statistical analysis of the historical data, or by comparison of the historical data with secondary data sets (e.g., manufacturer identified weight, emissions, etc. of make/model), or any combination thereof. The metrics may be, for example, vehicle tonnage, emissions, drive types, number, etc., over a section of the roadway per period of time.

In at least one embodiment, the analysis engine may determine a vehicle specific air quality index impact (“AQII”) metric for one or more vehicles, such that each vehicle specific AQII metric is uniquely associated with a respective vehicle. The vehicle specific AQII metric may characterize the impact of the associated vehicle on the Air Quality Index (“AQI”), or other gauge of air quality (e.g., air quality, greenhouse gases per part, smog per part, CO₂ per part, etc.), of one or more road segments. The vehicle-specific AQII metric may be a numeric value reflecting the amount by which the vehicle raised/lowered the AQI of the road segment(s).

The AQII may be determined from the traffic sensor generated data and/or other data associated with the vehicle for which the AQII is to be determined. The data used to determine the AQII may include, but is not limited to: vehicle make, model, body type, speed, weight, movement, specific emissions (CO₂/mi), etc. Such data may be directly provided by the traffic sensor(s), the server database(s), and/or may be identified from the recognition record(s).

In at least one embodiment, the analysis engine may determine a road segment specific AQII metric for one or more road segments, such that each road segment specific AQII metric is uniquely associated with a respective road segment. The road segment specific AQII metric may characterize the impact on the AQI, or other standard gauge of air quality, of the road segment that one or more vehicles have/had on the road segment. The vehicle-specific AQII metric may be a numeric value reflecting the amount by which the vehicle raised/lowered the AQI of the road segment(s).

One or more of the metrics may be dynamic metrics. That is to say that such metrics may be updated in real-time or in near real-time as additional traffic sensor generated data is received. It will be understood, however, that one or more of the metrics may reflect historic impact on the AQI over various time periods.

In at least one embodiment, the analysis engine may be configured to generate one or more AQII models reflecting one or more of the AQII metrics. The AQII models may visually and/or numerically depict the AQII metrics over one or more periods of time. For example, the AQII model may include one or more of: graphs, charts, tables, and the like.

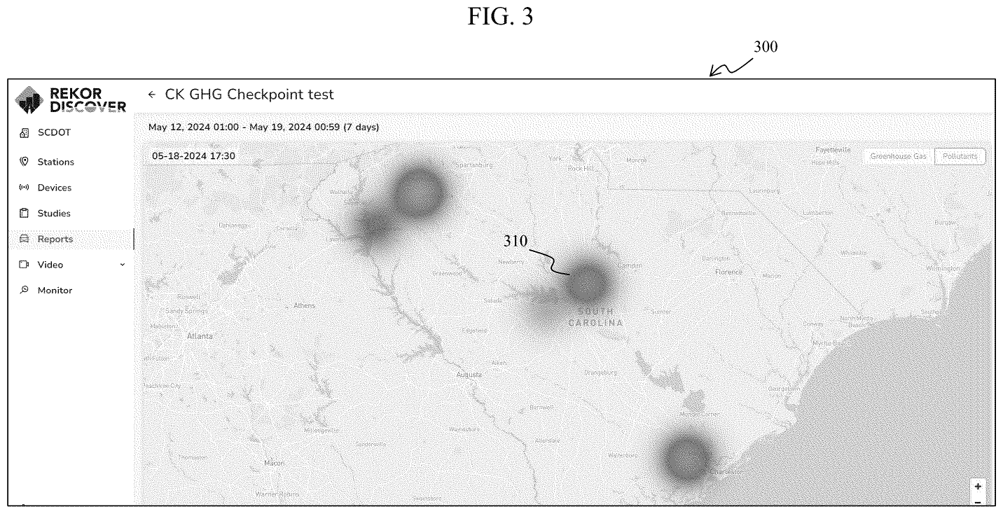

As shown in FIG. 3, in at least one embodiment, the AQII model comprises a visualized heat map 300 of the AQI over a geographic area in which the road segments are located. The heat map 300 may comprise one or more AQI impact hot-spots 310 indicating road segments where the AQI impact is above one or more thresholds.

In some embodiments, the AQII models can reflect the AQII metrics, as limited to selected parameters, such as, for example: time periods, vehicle characteristics, and traffic characteristics. For example, the road segment specific AQII model may reflect the AQI impact that long-haul shipping trucks had on that road segment during rush hours in December. Any other combination of time periods and/or vehicle/traffic characteristics may of course be similarly selectable parameters.

The system server 140 may store the metrics in the database for later retrieval, update, modification, deletion, etc. In at least one embodiment, the system server 140 transmits the metrics, via the network 160, to a third-party server (not shown), which may be one or more servers of law-enforcement (e.g., police, highway patrol, sheriff, etc.), civil service (e.g., department of transportation, municipality, etc.), and private (e.g., trucking company, security, etc.) entities.

The system server 140 may include one or more software applications, stored in the memory, which (when executed by the processor) configures the server computer to host and/or otherwise support one or more digital platforms 146. The digital platform 146 may be an online platform (e.g., a website) or a local platform (e.g., a closed computer network).

The digital platform may include a video management platform, which may be generally configured to permit users, via the user devices 180, to interact with video and other data stored by the system server 140. In particular, the video management platform 146 may support a graphical user interface 148 that permits users to select and retrieve video data (captured by the sensors 120) for video playback via the user device 180. In some embodiments, the video playback may be substantially up to real-time, or live-stream, video playback.

The graphical user interface 148 may also enable one or more playback functions, including but not limited to permitting users to pause, rewind and fast-forward the video playback. The graphical user interface 148 may further permit other interactions, which may include, for example, object recognition (e.g., license plate recognition, vehicle recognition, etc.), object tagging, video frame notations, data analytics, hit list comparison, and/or smart search capabilities.

The digital platform may include an air quality impact platform, which may be generally configured to permit users, via the user devices 180, to interact with the air quality impact metrics, models, and/or associated data stored by the system server 140. In particular, the air quality impact platform 146 may also support the graphical user interface 148, which may permit users to retrieve air quality impact metrics, models, and/or associated data for interactive display via the user device 180. In some embodiments, the metrics, models, and/or associated data may be substantially up to real-time.

The graphical user interface 148 may also enable one or more model interaction functions, including but not limited to permitting users to interact with the AQUI models. For example, where the AQUI model is a heat map visualization of the AQI over a time period, the graphical user interface 148 may permit users to pause, rewind and fast-forward the visualization. The graphical user interface 148 may further permit other interactions, which may include, for example, zooming in/out on or otherwise interacting with the model, selecting the time periods and/or other parameters reflected by the model, etc.

The user devices 180 are generally computing devices, and may include mobile (e.g., laptop computer, tablet computer, smartphone, PDA, wearable, etc.) or stationary (e.g., desktop computer, etc.), multi-purpose or dedicated, devices configured to communicate data and information with the system server 140. The user devices 180 may include components typically associated with such devices, such as one or more processors, physical memories, software instructions, data, displays, and interfaces. The user devices 180 may further include one or more software applications, stored in memory, which software applications, when executed by the processor, configures the user devices 180 to function as described herein. In particular, the user devices 180 are configured to allow the users to interact with the digital platforms 146, as described herein.

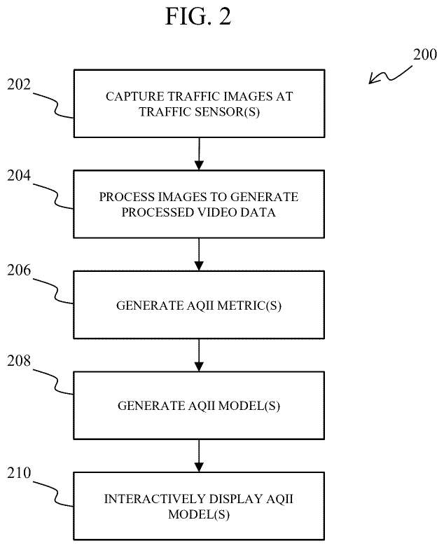

FIG. 2 is a flow-chart that represents an exemplary method 200 of operation for the traffic monitoring system 100 in accordance with one or more aspects of the invention.

At step 202, respective traffic sensors 120 capture images of vehicle traffic, namely, video images of passing vehicles, and generate video and/or image data therefrom. The traffic sensors 120 are preferably each positioned at various roadway locations where the vehicle traffic is to be monitored. The traffic sensors 120 are preferably positioned such that the captured images include the respective license plates of the passing vehicles, as well as other vehicle characteristics, e.g., vehicle type, class, make, model, color, year, drive type, license plate number, registration, trajectory, speed, location, etc., or any combination thereof.

At step 204, the video and/or image data captured by traffic sensors 120 is processed so as to generate the processed video data. The processed video data may include one or more recordation records. The controller 124 may utilize any image processing software suitable for this purpose.

At step 206, the traffic sensor 120 transmits the processed video data to the system server 140, which analyzes the processed video data to generate vehicle and/or traffic metrics, including: (a) the vehicle specific AQII metric for one or more vehicles, and (b) the road segment specific AQII metric for one or more road segments.

At step 208, the system server generates one or more AQII models reflecting one or more of the AQII metrics. The AQII models may visually and/or numerically depict the AQII metrics over one or more periods of time, and preferably includes the visualized heat map 300 (FIG. 3) of the AQI over a geographic area in which the road segments are located.

At step 210, the user accesses the digital platform 146 of the server system 140 so as to review, via the graphical user interface 148, the AQII model(s). Such access may be via one or more of the user devices 180 over the network connection.

In this manner, the AQI of the vehicles and road segments monitored by the traffic sensors 120 can be determined quickly, efficiently, inexpensively and without spectrophotometric sensors or tailpipe sensors.

The embodiments described in detail above are considered novel over the prior art and are considered critical to the operation of at least one aspect of the described systems, methods and/or apparatuses, and to the achievement of the above described objectives. The words used in this specification to describe the instant embodiments are to be understood not only in the sense of their commonly defined meanings, but to include by special definition in this specification: structure, material or acts beyond the scope of the commonly defined meanings. Thus, if an element can be understood in the context of this specification as including more than one meaning, then its use must be understood as being generic to all possible meanings supported by the specification and by the word or words describing the element.

The definitions of the words or drawing elements described herein are meant to include not only the combination of elements which are literally set forth, but all equivalent structure, material or acts for performing substantially the same function in substantially the same way to obtain substantially the same result. In this sense, it is therefore contemplated that an equivalent substitution of two or more elements may be made for any one of the elements described and its various embodiments or that a single element may be substituted for two or more elements.

Changes from the subject matter as viewed by a person with ordinary skill in the art, now known or later devised, are expressly contemplated as being equivalents within the scope intended and its various embodiments. Therefore, obvious substitutions now or later known to one with ordinary skill in the art are defined to be within the scope of the defined elements. This disclosure is thus meant to be understood to include what is specifically illustrated and described above, what is conceptually equivalent, what can be obviously substituted, and also what incorporates the essential ideas.

Furthermore, the functionalities described herein may be implemented via hardware, software, firmware or any combination thereof, unless expressly indicated otherwise. If implemented in software, the functionalities may be stored in a memory as one or more instructions on a computer readable medium, including any available media accessible by a computer that can be used to store desired program code in the form of instructions, data structures or the like. Thus, certain aspects may comprise a computer program product for performing the operations presented herein, such computer program product comprising a computer readable medium having instructions stored thereon, the instructions being executable by one or more processors to perform the operations described herein. It will be appreciated that software or instructions may also be transmitted over a transmission medium as is known in the art. Further, modules and/or other appropriate means for performing the operations described herein may be utilized in implementing the functionalities described herein.

The foregoing disclosure has been set forth merely to illustrate the invention and is not intended to be limiting. Since modifications of the disclosed embodiments incorporating the spirit and substance of the invention may occur to persons skilled in the art, the invention should be construed to include everything within the scope of the described embodiments and equivalents thereof.

1. A traffic monitoring system, comprising:
one or more traffic sensors configured to:
capture images of vehicles on a road segment,
generate traffic related data from the captured images, and
transmit the traffic related data to a server; and
the server configured to:
receive the traffic related data from the one or more traffic sensors,
analyze the traffic related data so as to generate an air quality index impact metric quantifying a change to an air quality index as indicated by the traffic related data.

2. The traffic monitoring system of claim 1, wherein the air quality index impact metric is vehicle specific so as to quantify the change to the air quality index due to a specific vehicle.

3. The traffic monitoring system of claim 1, wherein the air quality index impact metric is road segment specific so as to quantify the change to the air quality index of the road segment.

4. The traffic monitoring system of claim 1, wherein the air quality index impact metric is generated based on at least one of the following vehicle attributes: make, model, body type, speed, weight, movement, specific emissions (CO₂/mi).

5. The traffic monitoring system of claim 1, wherein the server is further configured to generate at least one model visually reflecting the air quality index impact metric.

6. The traffic monitoring system of claim 5, wherein the model comprises an air quality index heat map.

7. A traffic monitoring method, comprising:
capture images of vehicles on a road segment via one or more traffic sensors;
generating traffic related data from the captured images;
transmitting the traffic related data to a server; and
analyzing the traffic related data at the server so as to generate an air quality index impact metric quantifying a change to an air quality index as indicated by the traffic related data.

8. The traffic monitoring method of claim 7, wherein the air quality index impact metric is vehicle specific so as to quantify the change to the air quality index due to a specific vehicle.

9. The traffic monitoring method of claim 7, wherein the air quality index impact metric is road segment specific so as to quantify the change to the air quality index of the road segment.

10. The traffic monitoring method of claim 7, wherein the air quality index impact metric is generated based on at least one of the following vehicle attributes: make, model, body type, speed, weight, movement, specific emissions (CO₂/mi).

11. The traffic monitoring method of claim 7, the method further comprising:
generating at least one model visually reflecting the air quality index impact metric.

12. The traffic monitoring method of claim 11, wherein the model comprises an air quality index heat map.

13. A non-transitory computer-readable medium storing instructions that when executed by a computing device cause the computing device to:
receive from one or more traffic sensors traffic related data generated from captured images of vehicles on a road segment; and
analyze the traffic related data so as to generate an air quality index impact metric quantifying a change to an air quality index as indicated by the traffic related data.

14. The non-transitory computer-readable medium of claim 10, wherein the air quality index impact metric is vehicle specific so as to quantify the change to the air quality index due to a specific vehicle.

15. The non-transitory computer-readable medium of claim 10, wherein the air quality index impact metric is road segment specific so as to quantify the change to the air quality index of the road segment.

16. The non-transitory computer-readable medium of claim 10, wherein the air quality index impact metric is generated based on at least one of the following vehicle attributes: make, model, body type, speed, weight, movement, specific emissions (CO₂/mi).

17. The non-transitory computer-readable medium of claim 10, wherein executing the instructions further causes the computing device to:
generate at least one model visually reflecting the air quality index impact metric.

18. The non-transitory computer-readable medium of claim 17, wherein the model comprises an air quality index heat map.
